(19) United States
(12) Patent Application Publication
(10) Pub. No.: US 2025/0067712 A1
(43) Pub. Date: Feb. 27, 2025

(54) Oxygen Sensing Composition and Method of Use

(71) Applicant: Mohamed Magdy Ibrahim, Baltimore, MD (US)

(72) Inventor: Mohamed Magdy Ibrahim, Baltimore, MD (US)

(21) Appl. No.: 18/354,025

(22) Filed: Jul. 18, 2023

(51) Int. Cl. G01N 31/22; G01N 21/31; G01N 33/52

(52) U.S. Cl. CPC G01N 31/225; G01N 21/314; G01N 33/52

(57) ABSTRACT

A composition and method for measuring tissue oxygen tension which is implanted subcutaneously or inside the human body on an organ and measures tissue oxygen tension through fluorescence.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a composition and method for measuring tissue oxygen tension. In particular, it relates to a composition and method which is implanted subcutaneously or inside the human body on an internal organ and measures tissue oxygen tension through fluorescence.

### Description of Related Art

Tissue oxygen tension is the partial pressure of oxygen within the interstitial space of an organ bed. As it represents the balance between local oxygen delivery and consumption at any given time, it offers a ready monitoring capability to assess the adequacy of tissue perfusion relative to local demands.

The measurement of tissue oxygen tension (TOT) is currently measured by a number of methods. These include pulse oximetry, near infrared spectroscopy, and implantable fiber biosensors. While these sensors are effective, they are expensive and require elaborate electronics to measure tissue oxygen tension and continuous hands-on implementation. Furthermore, they are not designed for use in the home.

PolyHEMA or pHEMA is a hydrogel best known as poly-2-hydroxyethyl methacrylate. It is a soft, flexible, water-absorbing hydrogel plastic used to make soft contact lenses. It is a polymer of 2-hydroxyethyl methacrylate (HEMA), a clear liquid compound obtained by reacting methacrylic acid (CH₂═C[CH₃]CO₂H) with ethylene oxide or propylene oxide. HEMA can be shaped into a contact lens by being cast into a small, concave, spinning mold. Under the influence of heat or light and free-radical initiators, the HEMA polymerizes and its molecules link together to form long, multiple-unit chains. The HEMA repeating units of the polymer have the following chemical structure:

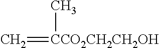

In most cases the polyHEMA chains are cross-linked into a complex three-dimensional network by another compound with which they are copolymerized. The cured plastic lens is hard but can absorb up to 60% of its weight in water, forming a soft hydrogel that has optical properties approaching those of hard contact lenses, yet is less irritating to the cornea of the eye.

There are a number of compositions that can measure oxygen using fluorescence. They are frequently utilized for deep tissue oxygen measurements making them hard to read the fluorescence without an invasive probe. Compositions include:
Palladium tetraphenyl-tetrabenzoporphyrin (PdTPTBP)
Platinum meso-tetraphenyltetrabenzoporphyrins PtTPTBP
Zinc 6-Aza-13,20,27-triphenyltetrabenzoporphyrin (ZnNTBP)
Zinc 6,13-Diaza-20,27-diphenyltetrabenzoporphyrin (ZnN2-cisTBP)
6-Aza-13,20,27-triphenyltetrabenzoporphyrin (H2NTBP)
6,13-Diaza-20,27-diphenyltetrabenzoporphyrin (H2N2-cisTBP)
Platinum(II) 6-Aza-13,20,27-triphenyltetrabenzoporphyrin (PtNTBP)
Palladium(II) 6-Aza-13,20,27-triphenyltetrabenzoporphyrin (PdNTBP)
Platinum(II) 6,13-Diaza-20,27-diphenyltetrabenzoporphyrin (PtN2-cisTBP)
Palladium(II) 6,13-Diaza-20,27-diphenyltetrabenzoporphyrin (PdN2-cisTBP)
Platinum(II)-meso-tetra-4-fluorophenyltetrabenzoporphyrin (PtTPTBPF)
Ruthenium(II) tris(4,7-diphenyl-1,10-phenanthroline) dichloride Ru(dpp)3Cl2
Ruthenium(II) tris(4,7-diphenyl-1,10-phenanthrolinedisulfonic acid) tetrasodium salt (Ru(dppds)3Na4

### BRIEF SUMMARY OF THE INVENTION

The present invention relates to a subcutaneously implantable oxygen sensing composition and method. It has been discovered that a pHEMA composition modified to be transparent and having an oxygen sensing compound imbedded therein can be placed subcutaneously under the skin of a mammal where tissue oxygen tension can be measured through the skin by a fluorescence sensing device, wherein the degree of fluorescence corresponds to the degree of oxygenation. There are several advantages of the novel composition including it being a transparent carrier, it contains superior mechanical properties including ability to stretch up to 300%, and the ability of the hydrogel to be dried before implantation making it easier to insert subcutaneously, wherein the hydrogel rehydrates after implantation. An advantage of the invention is the ability to do the treatment at home.

Accordingly, in one embodiment, there is an implantable oxygen sensing composition comprising:
a) a transparent hydrogel; and
b) an oxygen sensing composition which fluoresces in the presence of oxygen embedded in the transparent hydrogel.

In another embodiment, there is a method of measuring tissue oxygen tension in a mammal comprising:
a) selecting an implantable oxygen sensing composition comprising a transparent hydrogel and an oxygen sensing composition which fluoresces in the presence of oxygen embedded in the transparent hydrogel;
b) implanting the implantable oxygen sensing composition subcutaneously in the mammal; and
c) measuring the degree of fluorescence by measuring with a fluorescence reader.

### BRIEF DESCRIPTION OF THE DRAWINGS

FIG. 1 is flowchart of the present invention.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “implantable” and “implanting” and “implant” refers to a composition that is delivered under the skin into the subcutaneous tissue of a mammal or it can also be implanted inside the human body on an internal organ such as the liver, kidney, and the like. In one embodiment, it can be used in a human by surgery or injection and is a well-known procedure that is used to deliver a drug for an extended period of time. When used on an internal organ, light transmission to a reader is accomplished by a fiber optic (or the like) cable, which can be used to transmit light to the fluorescence reader outside of the body. When it is subcutaneously implanted, the oxygen sensing composition senses oxygen change and is measured by fluorescent light showing through the skin and the light is measured by a fluorescence reader which records light intensity changes.

As used herein, the term “oxygen sensing composition” refers to a composition which exhibits fluorescence in the presence of oxygen. Specifically, the intensity of the fluorescence is proportional to the amount of oxygen being sensed. Examples of such compositions include:
Palladium tetraphenyl-tetrabenzoporphyrin (PdTPTBP)
Platinum meso-tetraphenyltetrabenzoporphyrins PtTPTBP
Zinc 6-Aza-13,20,27-triphenyltetrabenzoporphyrin (ZnNTBP)
Zinc 6,13-Diaza-20,27-diphenyltetrabenzoporphyrin (ZnN2-cisTBP)
6-Aza-13,20,27-triphenyltetrabenzoporphyrin (H2NTBP)
6,13-Diaza-20,27-diphenyltetrabenzoporphyrin (H2N2-cisTBP)
Platinum(II) 6-Aza-13,20,27-triphenyltetrabenzoporphyrin (PtNTBP)
Palladium(II) 6-Aza-13,20,27-triphenyltetrabenzoporphyrin (PdNTBP)
Platinum(II) 6,13-Diaza-20,27-diphenyltetrabenzoporphyrin (PtN2-cisTBP)
Palladium(II) 6,13-Diaza-20,27-diphenyltetrabenzoporphyrin (PdN2-cisTBP)
Platinum(II)-meso-tetra-4-fluorophenyltetrabenzoporphyrin (PtTPTBPF)
Ruthenium(II) tris(4,7-diphenyl-1,10-phenanthroline) dichloride Ru(dpp)3Cl2
Ruthenium(II) tris(4,7-diphenyl-1,10-phenanthrolinedisulfonic acid) tetrasodium salt (Ru(dppds)3Na4

As used herein, the term “transparent hydrogel” refers to hydrogels that are three-dimensional network structures able to imbibe large amounts of water. Hydrogels do not typically dissolve due to chemical or physical cross-links and/or chain entanglements. They exist naturally in the form of polymer networks such as collagen or gelatin or it can be made synthetically. Many hydrogels can be made transparent. As an example, the HEMA hydrogel is a combination of 1-Butyl-3-methylimidasolium Chloride and 2-hydroxyethyl methacrylate that is degassed.

As used herein, the term “embedded in the transparent hydrogel” refers to the oxygen sensing compound being in the hydrogel, positioned such that it can still sense oxygen. The hydrogel is non-dissolving and will hold the oxygen sensing compound in place, thus making it is easy to implant the hydrogel subcutaneously so that it can sense oxygen.

As used herein, the term “measuring the degree of fluorescence” refers to fluorescence (also called phosphorescence) spectrometers used to measure emitted fluorescence. The spectrometer introduces ultra-violet or visible light using a photon source, like a laser, a xenon lamp, or LED (light emitting diode) lighting. The light passes through a monochromator that selects a specific wavelength, often using a diffraction grating. A diffraction grating is a plate of glass or metal ruled with very close parallel lines, producing a spectrum by diffraction and interference of light. The light that exits comes out at a specific angle depending on its wavelength.

### EXAMPLE

The transparent hydrogel with improved biomechanical properties is synthesized as follows:
a) 1-Butyl-3-methylimidazolium Chloride is mixed with 2-Hydroxyethyl Methacrylate (1:1 ratio) then degassed, then the oxygen sensing particles are added to the mixture
b) Polymerization of the mixture is done through adding Ammonium persulfate (0.5% W/V) and Tetramethylethylenediamine (0.05% W/V)
c) Compound is poured into a mold and is left to polymerize for 24 h: The polymerized gel is white in color and is opaque
d) The polymerized gel is then put in deionized water for a period of 48 hours, to allow for removal of 1-Butyl-3-methylimidazolium Chloride from the polymerized network to yield a transparent gel with favorable mechanical properties suitable for in-tissue use
e) The resulting hydrogel is elastic and can expand to 300% of the original size when hydrated, and it maintains the original size and shape when it is dehydrated.

### DRAWINGS

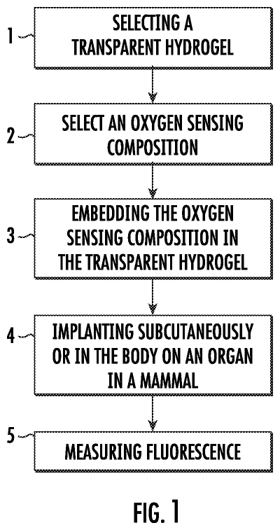

Now referring to the drawings, FIG. 1 is a flow chart of the method of the present invention. A transparent hydrogel is selected 1 and an oxygen sensing composition is selected 2. The oxygen sensing composition 2 is then embedded in the transparent hydrogel 3. The combined composition is then either implanted subcutaneously or implanted inside the body on an internal organ in a mammal 4. Then the amount of fluorescence of the oxygen sensing composition is measured 5.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. An implantable oxygen sensing composition comprising:
a) a transparent hydrogel; and
b) an oxygen sensing composition which fluoresces in the presence of oxygen embedded in the transparent hydrogel.

2. The implantable oxygen sensing composition according to claim 1, wherein the hydrogel is a HEMA hydrogel (polymer of 2-hydroxyethyl methacrylate) treated to be transparent.

3. The implantable oxygen sensing composition according to claim 2 wherein, the HEMA hydrogel is a combination of 1-Butyl-3-methylimidasolium Chloride and 2-hydroxyethyl methacrylate that is degassed.

4. The implantable oxygen sensing composition according to claim 1, wherein the fluorescence of the oxygen sensing composition can be connected to a fiber optic cable to deliver fluorescence to a fluorescence reader.

5. The implantable oxygen sensing composition according to claim 1, wherein the oxygen sensing composition can be subcutaneously implanted and read through the skin by a fluorescence reader.

6. The implantable oxygen sensing composition according to claim 1, wherein the oxygen sensing composition is selected from the group comprising:
a) Palladium tetraphenyl-tetrabenzoporphyrin (PdTPTBP);
b) Platinum meso-tetraphenyltetrabenzoporphyrins PtTPTBP;
c) Zinc 6-Aza-13,20,27-triphenyltetrabenzoporphyrin (ZnNTBP);
d) Zinc 6,13-Diaza-20,27-diphenyltetrabenzoporphyrin (ZnN2-cisTBP);
e) 6-Aza-13,20,27-triphenyltetrabenzoporphyrin (H2NTBP);
f) 6,13-Diaza-20,27-diphenyltetrabenzoporphyrin (H2N2-cisTBP);
g) Platinum(II) 6-Aza-13,20,27-triphenyltetrabenzoporphyrin (PtNTBP);
h) Palladium(II) 6-Aza-13,20,27-triphenyltetrabenzoporphyrin (PdNTBP);
i) Platinum(II) 6,13-Diaza-20,27-diphenyltetrabenzoporphyrin (PtN2-cisTBP);
j) Palladium(II) 6,13-Diaza-20,27-diphenyltetrabenzoporphyrin (PdN2-cisTBP);
k) Platinum(II)-meso-tetra-4-fluorophenyltetrabenzoporphyrin (PtTPTBPF);
l) Ruthenium(II) tris(4,7-diphenyl-1,10-phenanthroline) dichloride Ru(dpp)3Cl2; and
m) Ruthenium(II) tris(4,7-diphenyl-1,10-phenanthrolinedisulfonic acid) tetrasodium salt (Ru(dppds)3Na4.

7. A method of measuring tissue oxygen tension in a mammal comprising:
a) selecting an implantable oxygen sensing composition comprising a transparent hydrogel and an oxygen sensing composition which fluoresces in the presence of oxygen embedded in the transparent hydrogel;
b) implanting the implantable oxygen sensing composition in the mammal; and
c) measuring the degree of fluorescence by measuring with a fluorescence reader.

8. The method of measuring tissue oxygen tension in a mammal according to claim 7, wherein the hydrogel is a HEMA hydrogel treated to be transparent.

9. The method of measuring tissue oxygen tension in a mammal according to claim 8, wherein the HEMA hydrogel is a combination of 1-Butyl-3-methylimidasolium Chloride and 2-hydroxyethyl methacrylate that is degassed.

10. The method of measuring tissue oxygen tension in a mammal according to claim 7, wherein the oxygen sensing composition is selected from the group comprising:
a) Palladium tetraphenyl-tetrabenzoporphyrin (PdTPTBP);
b) Platinum meso-tetraphenyltetrabenzoporphyrins PtTPTBP;
c) Zinc 6-Aza-13,20,27-triphenyltetrabenzoporphyrin (ZnNTBP);
d) Zinc 6,13-Diaza-20,27-diphenyltetrabenzoporphyrin (ZnN2-cisTBP);
e) 6-Aza-13,20,27-triphenyltetrabenzoporphyrin (H2NTBP);
f) 6,13-Diaza-20,27-diphenyltetrabenzoporphyrin (H2N2-cisTBP);
g) Platinum(II) 6-Aza-13,20,27-triphenyltetrabenzoporphyrin (PtNTBP);
h) Palladium(II) 6-Aza-13,20,27-triphenyltetrabenzoporphyrin (PdNTBP);
i) Platinum(II) 6,13-Diaza-20,27-diphenyltetrabenzoporphyrin (PtN2-cisTBP);
j) Palladium(II) 6,13-Diaza-20,27-diphenyltetrabenzoporphyrin (PdN2-cisTBP);
k) Platinum(II)-meso-tetra-4-fluorophenyltetrabenzoporphyrin (PtTPTBPF);
l) Ruthenium(II) tris(4,7-diphenyl-1,10-phenanthroline) dichloride Ru(dpp)3Cl2; and
m) Ruthenium(II) tris(4,7-diphenyl-1,10-phenanthrolinedisulfonic acid) tetrasodium salt (Ru(dppds)3Na4.

11. The method of measuring tissue oxygen tension in a mammal according to claim 7, wherein the oxygen sensing composition is implanted subcutaneously.

12. The method of measuring tissue oxygen tension in a mammal according to claim 7, wherein the oxygen sensing composition is implanted on an internal organ and delivers fluorescence to the fluorescence reader via a fiber optic cable.
